(19) United States
(12) Patent Application Publication
(10) Pub. No.: US 2021/0301285 A1
(43) Pub. Date: Sep. 30, 2021

(54) Preparation of Combinatorial Libraries of DNA Constructs

(71) Applicant: Novozymes A/S, Bagsvaerd (DK)

(72) Inventors: Michael Lynge Nielsen, Birkeroed (DK); Nicholas Jochumsen, Lyngby (DK); José Arnau, Hellerup (DK)

(73) Assignee: Novozymes A/S, Bagsvaerd (DK)

(21) Appl. No.: 17/265,092

(22) PCT Filed: Jul. 19, 2019
(86) PCT No.: PCT/EP2019/069495

§ 371 Date: Feb. 1, 2021

Related U.S. Application Data
(60) Provisional application No. 62/713,838, filed on Aug. 2, 2018.

(30) Foreign Application Priority Data
Aug. 28, 2018 (EP) 18191207.2

(51) Int. Cl. C12N 15/10; C12N 15/81; C12N 15/90

(52) U.S. Cl. CPC C12N 15/1051; C12N 15/81; C12N 2830/36; C12N 2830/42; C12N 15/905

(57) ABSTRACT

Means and methods for preparing combinatorial libraries of DNA constructs, in particular expression cassettes, including nucleic acid constructs, expression vectors, host cells, methods for preparing host cells, and methods for producing polypeptides of interest, whereby the expression comprises an first intron and a second intron on either side of the polynucleotide to be expressed and a promoter and a terminator. Also claimed is a method of constructing eukaryotic host cells in which the cells are contacted with three polynucleotides and in which the first and second and the second and third are pairwise capable of homologous recombination and of subsequent formation of introns.

### REFERENCE TO SEQUENCE LISTING

This application contains a Sequence Listing in computer readable form. The computer readable form is incorporated herein by reference.

### FIELD OF THE INVENTION

The present invention relates to means and methods for preparing combinatorial libraries of DNA constructs, in particular expression cassettes, including nucleic acid constructs, expression vectors, host cells, methods for preparing host cells, and methods for producing polypeptides of interest

### BACKGROUND OF THE INVENTION

Within the biotech industry, production of relevant polypeptides in general requires optimization of all constituents of the expression system to ensure the highest possible yield. An important aspect of this is optimization of the expression cassette, which include codon optimization of the coding sequence as well as elucidation of the optimal configuration of the control sequences that direct expression of the coding sequence.

Historically, optimization of expression cassettes has been performed using trial-and-error based methodologies that involves a compromise between cassette diversity and screening time. However, a combinatorial approach for construction expression cassette libraries would enable high-throughput screening while maintaining high cassette diversity and without compromising screening time and product yield.

### SUMMARY OF THE INVENTION

The present invention is based on the surprising and inventive finding that introns may be used to generate modular DNA elements useful for in vivo generation of combinatorial libraries of DNA constructs.

In a first aspect, the present invention relates to a nucleic acid construct comprising in a 5′ to 3′ direction a promoter, a first intron, a polynucleotide encoding a polypeptide of interest, a second intron, and a transcription terminator, wherein the promoter, the first intron, the polynucleotide encoding a polynucleotide of interest, the second intron, and the transcription terminator are operably linked.

In a second aspect, the present invention relates to an expression vector comprising a nucleic acid construct according to the first aspect.

In a third aspect, the present invention relates to a eukaryotic host cell comprising in its genome a nucleic acid construct according to the first aspect or an expression vector according to the second aspect.

In a fourth aspect, the present invention relates to a method for constructing a eukaryotic host cell, the method comprising transforming a eukaryotic cell with:

a) a first polynucleotide comprising in a ′5 to 3 direction a promoter and a first DNA sequence;

b) a second polynucleotide comprising in a ′5 to ′3 direction a second DNA sequence, a coding sequence of a polypeptide of interest, and a third DNA sequence; and

c) a third polynucleotide comprising in a ′5 to 3′ direction a fourth DNA sequence and a transcription terminator;

wherein the first, second, and third polynucleotides are operably linked, wherein the first and second DNA sequences and the third and fourth DNA sequences are pairwise capable of homologous recombination and subsequent formation of introns, and wherein the resulting introns are capable of RNA splicing upon transcription.

In a fifth aspect, the present invention relates to a method for producing a polypeptide of interest, the method comprising the steps of:

a) providing a eukaryotic host cell according to the third aspect OR prepared by a method according to the fourth aspect;

b) cultivating said host cell under conditions conducive for expression of the polypeptide of interests; and, optionally

c) recovering the polypeptide of interest.

### BRIEF DESCRIPTION OF DRAWINGS

FIG. 1 shows that the coding sequence of a lipase can be amplified as two PCR fragments and assembled in vivo using different introns having 30 bp 5′ and 3′ flank homologous to PCR1 and PCR2.

FIG. 2 shows SDS-PAGE (panel A, bottom) of culture supernatant from strains containing 21 different introns (SEQ ID NO: 1-21) as well as relevant controls, and lipase units (LU, panel B, top) of the corresponding supernatants in panel A. The intron number # is shown above each band on the SDS-PAGE, with “1” corresponding to SEQ ID NO: 1, etc. “pyrG” is a well-known intron from A. nidulans pyrG (AN6157), “8k” is a control strain without an insertion, “B” is the background strain with a blank expression cassette, and “%” is a medium control.

FIG. 3 shows the principle of joining several DNA fragments using three different introns and two non-intron linkers.

FIG. 4 shows SDS-PAGE gel of supernatants from strains cultivated for five days in YPM. Experiments 1-3 differ in the use of different promotors (P1-P3). For each experiment, four strains were selected and cultivated (lanes 1 to 4 in each case). “C1” is lipase gene with intron #15 (SEQ ID NO: 15, from Example 1); “C2” is lipase gene with intron #21 (SEQ ID NO: 21, from Example 1) and “C3” is lipase gene with no intron but with a different promotor.

FIG. 5 shows lipase vector construction. Vector 1 and vector 2 had a single intron within the lipase gene. Vector 3 had no introns. Vector 4 had two introns flanking the region of the polynucleotide encoding the signal peptide and pro-peptide.

FIG. 6 shows SDS-PAGE of supernatants from multi-copy strains grown for five days. The copy number of the lipase gene is indicated above each lane.

FIG. 7 shows the principle of joining several DNA fragments using three different introns and two non-intron linkers. Here, three different promotors are mixed simultaneously allowing the construction of three different types of transformants simultaneously and creation of a combinatorial library.

FIG. 8 shows SDS-page of supernatants from strains grown for five days at 30° C. “C1” is lipase gene with intron #15 (SEQ ID NO: 15), “C2” is lipase gene with intron #21 (SEQ ID NO: 21, and “C3” is lipase gene with no intron but with a different promotor.

FIG. 9 shows sequencing of strain #13 from Example 4, revealing an insertion of thirty-three nucleotides within the sequence of intron #8 (SEQ ID NO:26; SEQ ID NO:27).

FIG. 10 shows cases of insertions and deletion observed in strains from Example 2 and Example 4. The strains were still capable of producing lipase, indicating that the introns are functional despite the mutations (SEQ ID NO:28; SEQ ID NO:29; SEQ ID NO:30; SEQ ID NO:31).

FIG. 11 shows a single base deletion within intron #7 (SEQ ID NO: 7) as observed in strain #4 from Example 2 (SEQ ID NO:32; SEQ ID NO:33).

FIG. 12 shows the matrix for constructing six different codon variants of lipase (dotted box) using introns as linkers.

FIG. 13 shows SDS-PAGE of supernatants from strains grown for five days at 30° C. Each variant is represented, demonstrating that the matrix cloning principle results in production of lipase. The reference (REF) is C3 from Example 2, the lipase gene with no intron but with a different promotor. The # numbers indicate the particular gene variant used for the matrix cloning.

### DEFINITIONS

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other.

Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

### DETAILED DESCRIPTION OF THE INVENTION

The present invention is based on the surprising and inventive finding that introns may be used to generate combinatorial libraries of nucleic acid constructs. As shown in the Examples disclosed herein, introns may be used to join polynucleotides of interest in a predefined order to form nucleic acid constructs of interest. Upon transcription of the nucleic acid constructs, the introns are removed from the resulting mRNA by the mRNA processing machinery of the host cell.

The nucleic acid constructs are assembled from modular DNA elements that each contain a cargo sequence flanked by intron-forming sequences on one or both sides. The cargo sequence may in principle contain any polynucleotide sequence of interest. In the context of expression cassettes, relevant cargo sequences include, but is not limited to, promoters, polynucleotides encoding signal peptides, polynucleotides encoding polypeptides of interest, and transcription terminators.

The modular DNA elements are combined in vivo upon transformation of a suitable host cell and subsequent homologous recombination between the intron-forming sequences, resulting in formation of nucleic acid constructs that contain the cargo sequences separated by functional introns. The order of cargo sequences in the nucleic acid constructs is determined by ensuring controlled pairwise recombination of the intron-forming sequences. By varying the cargo sequences of the modular elements, a combinatorial library of nucleic acid constructs may be generated. By cultivating the host cells under suitable conditions, the nucleic acid constructs may be expressed and the effects of individual cargo sequences on the expression output may be evaluated. Thus, in the context of expression cassettes, the present invention is suitable for identifying the optimal configuration of promoter, signal peptide, coding sequence and terminator.

Thus, in a first aspect, the present invention relates to a nucleic acid construct comprising in a 5′ to 3′ direction a promoter, a first intron, a polynucleotide encoding a polypeptide of interest, a second intron, and a transcription terminator, wherein the promoter, the first intron, the polynucleotide encoding a polypeptide of interest, the second intron, and the transcription terminator are operably linked.

A nucleic acid construct according to the first aspect is suitable for screening and evaluation of combinations of promoters, polynucleotide encoding a polypeptide of interest, and transcription terminators. However, in some cases, it may also be valuable to include signal peptides in the screening setup, e.g., if the polypeptide of interest is secreted.

Thus, in a preferred embodiment of the first aspect, the nucleic acid construct further comprises a polynucleotide encoding a signal peptide and a third intron, wherein the polynucleotide encoding a signal peptide and the third intron are operably linked to and located between the first intron and the coding sequence of a polypeptide of interest.

Alternatively stated, in a preferred embodiment of the first aspect, the nucleic acid comprises in a 5′ to 3′ direction a promoter, a first intron, a polynucleotide sequence encoding a signal peptide, a second intron, a coding sequence of a polypeptide of interest, a third intron, and a transcription terminator, wherein the promoter, the first intron, the polynucleotide encoding a signal peptide, the second intron, the polynucleotide encoding a polypeptide of interest, the third intron, and the transcription terminator are operably linked.

Any functional intron capable of undergoing RNA splicing is useful with the present invention. Introns of the invention may be naturally occurring introns, variants or fragments of naturally occurring introns, or synthetic introns. Preferably, the introns are heterologous to a host cell comprising a nucleic acid construct of the invention.

In a preferred embodiment, the introns are different and individually comprise no more than 200 nucleotides, i.e., no more than 175, 150, 125, 100, 90, 80, 70, 60, or 50 nucleotides.

In a preferred embodiment, the introns comprise a GT donor site and/or an AG acceptor site.

In a preferred embodiment, the introns are capable of RNA splicing upon transcription.

In a preferred embodiment, the introns are individually selected from the group consisting of SEQ ID NO: 1-21.

In a preferred embodiment, the first intron is located between the promoter and the start codon of the polynucleotide encoding a polypeptide of interest.

In a preferred embodiment, the second intron is located between the stop codon of the polynucleotide encoding a polypeptide of interest and the transcription terminator.

The nucleic acid constructs of the invention may further comprise linker polynucleotides for inserting the nucleic acid construct into an expression vector or into the genome of a host cell.

In a preferred embodiment, the nucleic acid construct further comprises a linker polynucleotide located upstream of the promoter.

In a preferred embodiment, the nucleic acid construct further comprises a linker polynucleotide located downstream of the terminator

### Nucleic Acid Constructs

The first aspect of the present invention relates to nucleic acid constructs comprising a polynucleotide encoding a polypeptide of interest operably linked to one or more control sequences that direct the expression of the polynucleotide in a suitable host cell under conditions compatible with the control sequences.

In a preferred embodiment, the polypeptide of interest comprises or consists of an enzyme; preferably the enzyme is selected from the group consisting of hydrolase, isomerase, ligase, lyase, oxidoreductase, or transferase; more preferably the enzyme is selected from the group consisting of aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, nuclease, oxidase, pectinolytic enzyme, peroxidase, phosphodiesterase, phytase, polyphenoloxidase, proteolytic enzyme, ribonuclease, transglutaminase, xylanase, and beta-xylosidase.

The polynucleotide encoding a polypeptide of interest may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including variant, truncated, and hybrid promoters, and may be obtained from genes encoding extracellular or intracellular polypeptides either homologous or heterologous to the host cell.

In a preferred embodiment, the promoter is a heterologous promoter; preferably the promoter is a fungal promoter.

In a preferred embodiment, the fungal promoter is a filamentous fungal promotor. Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Dania (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus those phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and variant, truncated, and hybrid promoters thereof. Other promoters are described in U.S. Pat. No. 6,011,147.

In a preferred embodiment, the fungal promoter is a yeast promoter. In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

In a preferred embodiment, the terminator is a fungal terminator.

In a preferred embodiment, the fungal terminator is a filamentous fungal terminator. Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor.

In a preferred embodiment, the fungal terminator is a yeast terminator. Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be a leader, a nontranslated region of an mRNA that is important for translation by the host cell. The leader is operably linked to the 5′-terminus of the polynucleotide encoding the polypeptide of interest. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

In a preferred embodiment, the signal peptide is a fungal signal peptide.

In a preferred embodiment, the fungal signal peptide is a filamentous fungal signal peptide. Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

In a preferred embodiment, the fungal signal peptide is a yeast signal peptide. Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. For fungal host cells, the propeptide coding sequence may be obtained from the genes for Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide of interest would be operably linked to the regulatory sequence.

### Expression Vectors

In a second aspect, the present invention also relates to recombinant expression vectors comprising a nucleic acid construct of the present invention.

The recombinant expression vector may be any vector (e.g., a plasmid or virus) that can be conveniently subjected to recombinant DNA procedures and can bring about expression of the nucleic acid construct. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Suitable markers for yeast host cells include, but are not limited to, ADE2, HIS3, LEU2, LYS2, MET3, TRP1, and URA3. Selectable markers for use in a filamentous fungal host cell include, but are not limited to, adeA (phosphoribosylaminoimidazole-succinocarboxamide synthase), adeB (phosphoribosyl-aminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one embodiment, the dual selectable marker is an hph-tk dual selectable marker system.

The vector preferably contains an element(s) that permits integration of the vector into the host cell's genome or autonomous replication of the vector in the cell independent of the genome.

For integration into the host cell genome, the vector may rely on the polynucleotide sequences of the nucleic acid construct or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANSI (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a nucleic acid construct of the present invention may be inserted into a host cell to increase production of a polypeptide. An increase in the copy number of the nucleic acid construct can be obtained by integrating at least one additional copy of the construct into the host cell genome or by including an amplifiable selectable marker gene with the construct where cells containing amplified copies of the selectable marker gene, and thereby additional copies of the construct, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

In a third aspect, the present invention also relates to recombinant host cells comprising a nucleic acid construct of the present invention. A construct or vector comprising a construct is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the polynucleotide encoding a polypeptide of interest and its source.

The host cell may be any cell useful in the recombinant production of a polypeptide interest. Preferably, the host cell is a eukaryotic host cell, such as a mammalian, insect, plant, or fungal cell.

In a preferred embodiment, the host cell is a fungal host cell. The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

In a preferred embodiment, the fungal host cell is a yeast host cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

Preferably, the yeast host cell is a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

In a preferred embodiment, the fungal host cell is a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

Preferably, the filamentous fungal host cell is an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

More preferably, the filamentous fungal host cell is an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonaturn, Coprinus cinereus, Coriolus hirsutus, Fusarium bactridioides, Fusarium cerealis, Fusarium crookwellense, Fusarium culmorum, Fusarium graminearum, Fusarium graminurn, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenaturn, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianurn, Trichoderma koningii, Trichoderma longi brachiatum, Trichoderma reesei, or Trichoderma viride cell. Most preferably, the filamentous fungal host cell is an Aspergillus niger, Aspergillus oryzae, Fusarium venenatum, or Trichoderma reesei cell.

Host cells of the present invention may be prepared by transforming a suitable cell with the modular DNA elements necessary for forming a nucleic acid construct of the invention.

Thus, in a fourth aspect, the present invention relates to a method for preparing a eukaryotic host cell, the method comprising transforming a eukaryotic cell with:

a) a first polynucleotide comprising in a ′5 to 3 direction a promoter and a first DNA sequence;

b) a second polynucleotide comprising in a ′5 to ′3 direction a second DNA sequence, a polynucleotide encoding a polypeptide of interest, and a third DNA sequence; and

c) a third polynucleotide comprising in a ′5 to 3′ direction a fourth DNA sequence and a terminator;

wherein the first, second, and third polynucleotides are operably linked, wherein the first and second DNA sequences and the third and fourth DNA sequences are pairwise capable of homologous recombination and subsequent formation of introns, and wherein the resulting introns are capable of RNA splicing upon transcription.

Preparation of host cells comprising a nucleic acid construct comprising a signal peptide requires transformation with an additional modular DNA element.

Thus, in a preferred embodiment, the host cell is further transformed with a fourth polynucleotide comprising in a 5′ to 3′ direction a fifth DNA element, a polynucleotide encoding a signal peptide, and a sixth DNA element, wherein the first, second, third, and fourth polynucleotides are operably linked, wherein the first and fifth DNA sequences, the sixth and second DNA sequences, and the third and fourth DNA sequences are pairwise capable of homologous recombination and subsequent formation of introns, and wherein the resulting introns are capable of RNA splicing upon transcription.

Alternatively stated, in a preferred embodiment, the present invention relates to a method for preparing a eukaryotic host cell, the method comprising transforming a eukaryotic cell with:

a) a first polynucleotide comprising in a ′5 to 3 direction a promoter and a first DNA sequence;

b) a second polynucleotide comprising in a ′5 to ′3 direction a second DNA sequence, a polynucleotide encoding a signal peptide, and a third DNA sequence;

c) a third polynucleotide comprising in a ′5 to 3′ direction a fourth DNA sequence, a polynucleotide encoding a polypeptide of interest, and a fifth DNA sequence; and

d) a fourth polynucleotide comprising in a ′5 to 3′ direction a sixth DNA sequence and a transcription terminator;

wherein the first, second, third, and fourth polynucleotides are operably linked, wherein the first and second DNA sequences, the third and fourth DNA sequences, and the fifth and sixth DNA sequences are pairwise capable of homologous recombination and subsequent formation of introns, and wherein the resulting introns are capable of RNA splicing upon transcription.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

### Methods of Production

In a fifth aspect, the present invention also relates to methods of producing a polypeptide of interest, the method comprising:

a) providing a eukaryotic host cell according to the third aspect of the invention or prepared by a method according to the fourth aspect of the present invention;

b) cultivating said host cell under conditions conducive for expression of the polypeptide of interests; and, optionally

c) recovering the polypeptide of interest.

The host cells are cultivated in a nutrient medium suitable for production of the polypeptide of interest using methods known in the art. For example, the cells may be cultivated by shake flask cultivation, or small-scale or large-scale fermentation (including continuous, batch, fed-batch, or solid state fermentations) in laboratory or industrial fermentors in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide of interest is secreted into the nutrient medium, the polypeptide can be recovered directly from the medium. If the polypeptide of interest is not secreted, it can be recovered from cell lysates.

The polypeptide of interest may be detected using methods known in the art that are specific for that sort of polypeptide. These detection methods include, but are not limited to, use of specific antibodies, formation of an enzyme product, or disappearance of an enzyme substrate. For example, an enzyme assay may be used to determine the activity of the polypeptide.

The polypeptide of interest may be recovered using methods known in the art. For example, the polypeptide may be recovered from the nutrient medium by conventional procedures including, but not limited to, collection, centrifugation, filtration, extraction, spray-drying, evaporation, or precipitation. In one embodiment, a fermentation broth comprising the polypeptide of interest is recovered.

The polypeptide of interest may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

In an alternative aspect, the polypeptide of interest is not recovered, but rather a host cell of the present invention expressing the polypeptide is used as a source of the polypeptide.

The present invention is further illustrated by the following list of preferred embodiments.

### Preferred Embodiments

1) A nucleic acid construct comprising in a 5′ to 3′ direction a promoter, a first intron, a polynucleotide encoding a polypeptide of interest, a second intron, and a transcription terminator, wherein the promoter, the first intron, the polynucleotide encoding a polynucleotide of interest, the second intron, and the transcription terminator are operably linked.
2) The nucleic acid construct according to embodiment 1, which further comprises a polynucleotide encoding a signal peptide and a third intron, wherein the polynucleotide encoding a signal peptide and the third intron are operably linked to and located between the first intron and the polynucleotide encoding a polypeptide of interest.
3) The nucleic acid construct according to any of the preceding embodiments, wherein the promoter is a heterologous promoter.
4) The nucleic acid construct according to any of the preceeding embodiments, wherein the polypeptide of interest comprises or consists of an enzyme; preferably the enzyme is selected from the group consisting of hydrolase, isomerase, ligase, lyase, oxidoreductase, or transferase; more preferably the enzyme is selected from the group consisting of aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, nuclease, oxidase, pectinolytic enzyme, peroxidase, phosphodiesterase, phytase, polyphenoloxidase, proteolytic enzyme, ribonuclease, transglutaminase, xylanase, and beta-xylosidase.
5) The nucleic acid construct according to any of the preceding embodiments, wherein the introns are different and individually comprise no more than 200 nucleotides, i.e., no more than 175, 150, 125, 100, 90, 80, 70, 60, or 50 nucleotides.
6) The nucleic acid construct according to any of the preceding embodiments, wherein the introns comprise a GT donor site and/or an AG acceptor site.
7) The nucleic acid construct according to any of the preceding embodiments, wherein the introns are capable of RNA splicing upon transcription.
8) The nucleic acid construct according to any of the preceding embodiments, wherein the introns are heterologous to the host cell.
9) The nucleic acid construct according to any of the preceding embodiments, wherein the introns are individually selected from the group consisting of SEQ ID NO: 1-21.
10) The nucleic acid construct according to any of the preceding embodiments, wherein the first intron is located between the promoter and the start codon of the polynucleotide encoding a polypeptide of interest.
11) The nucleic acid construct according to any of the preceding embodiments, wherein the second intron is located between the stop codon of the polynucleotide encoding a polypeptide of interest and the transcription terminator.
12) The nucleic acid construct according to any of the preceding embodiments, wherein the expression cassette further comprises a linker polynucleotide located upstream of the promoter.
13) The nucleic acid construct according to any of the preceding embodiments, wherein the expression cassette further comprises a linker polynucleotide located downstream of the terminator.
14) An expression vector comprising a nucleic acid construct comprising in a 5′ to 3′ direction a promoter, a first intron, a polynucleotide encoding a polypeptide of interest, a second intron, and a transcription terminator, wherein the promoter, the first intron, the polynucleotide encoding a polynucleotide of interest, the second intron, and the transcription terminator are operably linked.
15) The expression vector according to embodiment 14, wherein the nucleic acid construct further comprises a polynucleotide encoding a signal peptide and a third intron, wherein the polynucleotide encoding a signal peptide and the third intron are operably linked to and located between the first intron and the polynucleotide encoding a polypeptide of interest.
16) The expression vector according to any of embodiments 14-15, wherein the promoter is a heterologous promoter.
17) The expression vector according to any of embodiments 14-16, wherein the polypeptide of interest comprises or consists of an enzyme; preferably the enzyme is selected from the group consisting of hydrolase, isomerase, ligase, lyase, oxidoreductase, or transferase; more preferably the enzyme is selected from the group consisting of aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, nuclease, oxidase, pectinolytic enzyme, peroxidase, phosphodiesterase, phytase, polyphenoloxidase, proteolytic enzyme, ribonuclease, transglutaminase, xylanase, and beta-xylosidase.
18) The expression vector according to any of embodiments 14-17, wherein the introns are different and individually comprise no more than 200 nucleotides, i.e., no more than 175, 150, 125, 100, 90, 80, 70, 60, or 50 nucleotides.
19) The expression vector according to any of embodiments 14-18, wherein the introns comprise a GT donor site and/or an AG acceptor site.
20) The expression vector according to any of embodiments 14-19, wherein the introns are capable of RNA splicing upon transcription.
21) The expression vector according to any of embodiments 14-20, wherein the introns are heterologous to the host cell.
22) The expression vector according to any of embodiments 14-21, wherein the introns are individually selected from the group consisting of SEQ ID NO: 1-21.
23) The expression vector according to any of embodiments 14-22, wherein the first intron is located between the promoter and the start codon of the polynucleotide encoding a polypeptide of interest.
24) The expression vector according to any of embodiments 14-23, wherein the second intron is located between the stop codon of the polynucleotide encoding a polypeptide of interest and the transcription terminator.
25) The expression vector according to any of embodiments 14-24, wherein the expression cassette further comprises a linker polynucleotide located upstream of the promoter.
26) The expression vector according to any of embodiments 14-25, wherein the expression cassette further comprises a linker polynucleotide located downstream of the terminator.
27) A eukaryotic host cell comprising in its genome a nucleic acid construct comprising in a 5′ to 3′ direction a promoter, a first intron, a polynucleotide encoding a polypeptide of interest, a second intron, and a transcription terminator, wherein the promoter, the first intron, the coding sequence, the second intron, and the transcription terminator are operably linked; OR an expression vector comprising a nucleic acid construct comprising in a 5′ to 3′ direction a promoter, a first intron, a polynucleotide encoding a polypeptide of interest, a second intron, and a transcription terminator, wherein the promoter, the first intron, the polynucleotide encoding a polynucleotide of interest, the second intron, and the transcription terminator are operably linked.
28) The eukaryotic host cell according to embodiment 27, wherein the nucleic acid construct further comprises a polynucleotide sequence encoding a signal peptide and a third intron, and wherein the polynucleotide sequence encoding a signal peptide and the third intron are operably linked to and located between the first intron and the coding sequence of a polypeptide of interest
29) The eukaryotic host cell according to any of embodiments 27-28, said host cell being a mammalian, plant, or fungal host cell; preferably said host cell is a fungal host cell.
30) The eukaryotic host cell of embodiment 29, wherein the fungal host cell is a yeast host cell; preferably the yeast host cell is selected from the group consisting of Candida, Hansenula, Kluyveromyces, Pichia, Saccharomyces, Schizosaccharomyces, and Yarrowia cell; more preferably the yeast host cell is selected from the group consisting of Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, and Yarrowia lipolytica cell.
31) The eukaryotic host cell of embodiment 29, wherein the fungal host cell is a filamentous fungal host cell; preferably the filamentous fungal host cell is selected from the group consisting of Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, and Trichoderma cell; more preferably the filamentous fungal host cell is selected from the group consisting of Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianurn, Trichoderma koningii, Trichoderma longibrachiatum, Trichoderma reesei, and Trichoderma viride cell; most preferably the filamentous host cell is selected from the group consisting of Aspergillus niger, Aspergillus oryzae, Fusarium venenatum, and Trichoderma reesei.
32) The eukaryotic host cell according to any of embodiments 27-31, wherein the polypeptide of interest comprises or consists of an enzyme; preferably the enzyme is selected from the group consisting of hydrolase, isomerase, ligase, lyase, oxidoreductase, or transferase; more preferably the enzyme is selected from the group consisting of aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, nuclease, oxidase, pectinolytic enzyme, peroxidase, phosphodiesterase, phytase, polyphenoloxidase, proteolytic enzyme, ribonuclease, transglutaminase, xylanase, and beta-xylosidase.
33) The eukaryotic host cell according to any of embodiments 27-32, wherein the introns are different and individually comprise no more than 200 nucleotides, i.e., no more 175, 150, 125, 100, 90, 80, 70, 60, or 50 nucleotides.
34) The eukaryotic host cell according to any of embodiments 27-33, wherein the introns comprises a GT donor site and/or an AG acceptor site.
35) The eukaryotic host cell according to any of embodiments 27-34, wherein the introns are capable of RNA splicing upon transcription.
36) The eukaryotic host cell according to any of embodiments 27-35, wherein the introns are heterologous to the host cell.
37) The eukaryotic host cell according to any of embodiments 27-36 wherein the introns are individually selected from the group consisting of SEQ ID: NO 1-21.
38) The eukaryotic host cell according to any of embodiments 27-37, wherein the first intron is located between the promoter and the start codon of the polynucleotide encoding a polypeptide of interest.
39) The eukaryotic host cell according to any of embodiments 27-38, wherein the second intron is located between the stop codon of the polynucleotide encoding a polypeptide of interest and the transcription terminator.
40) The eukaryotic host cell according to any of embodiments 27-39, wherein the expression cassette further comprises a linker polynucleotide located upstream of the promoter.
41) The eukaryotic host cell according to any of embodiments 27-40, wherein the expression cassette further comprises a linker polynucleotide located downstream of the terminator.
42) A method for constructing a eukaryotic host cell, the method comprising transforming a eukaryotic cell with:

a) a first polynucleotide comprising in a ′5 to 3 direction a promoter and a first DNA sequence;

b) a second polynucleotide comprising in a ′5 to ′3 direction a second DNA sequence, a coding sequence of a polypeptide of interest, and a third DNA sequence; and

c) a third polynucleotide comprising in a ′5 to 3′ direction a fourth DNA sequence and a transcription terminator;

wherein the first, second, and third polynucleotides are operably linked, wherein the first and second DNA sequences and the third and fourth DNA sequences are pairwise capable of homologous recombination and subsequent formation of introns, and wherein the resulting introns are capable of RNA splicing upon transcription.

43) The method according to embodiment 42, wherein the host cell is further transformed with a fourth polynucleotide comprising in a 5′ to 3′ direction a fifth DNA sequence, a polynucleotide encoding a signal peptide, and a sixth DNA sequence;

wherein the first, second, third, and fourth polynucleotides are operably linked, wherein the first and fifth DNA sequences, the sixth and second DNA sequences, and the third and fourth DNA sequences are pairwise capable of homologous recombination and subsequent formation of introns, and wherein the resulting introns are capable of RNA splicing upon transcription.

44) The method according to any of embodiments 42-43, wherein the eukaryotic cell is a fungal cell.
45) The method according to embodiment 44, wherein the fungal cell is a yeast cell; preferably the yeast cell is selected from the group consisting of Candida, Hansenula, Kluyveromyces, Pichia, Saccharomyces, Schizosaccharomyces, and Yarrowia cell; more preferably the yeast cell is selected from the group consisting of Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, and Yarrowia lipolytica cell.
46) The method according to embodiment 44, wherein the fungal cell is a filamentous fungal cell; preferably the filamentous fungal cell is selected from the group consisting of Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, and Trichoderma cell; more preferably the filamentous fungal cell is selected from the group consisting of Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, and Trichoderma viride cell; most preferably the filamentous fungal cell is selected from the group consisting of Aspergillus niger, Aspergillus oryzae, Fusarium venenatum, and Trichoderma reesei.
47) A method for producing a polypeptide of interest, the method comprising the steps of:

a) providing a eukaryotic host cell according to any of embodiments 27-41 OR prepared by a method according to any of embodiments 42-46;

b) cultivating said host cell under conditions conducive for expression of the polypeptide of interests; and, optionally

c) recovering the polypeptide of interest.

48) The eukaryotic host cell according to embodiment 44, wherein the polypeptide of interest comprises or consists of an enzyme; preferably the enzyme is selected from the group consisting of hydrolase, isomerase, ligase, lyase, oxidoreductase, or transferase; more preferably the enzyme is selected from the group consisting of aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, nuclease, oxidase, pectinolytic enzyme, peroxidase, phosphodiesterase, phytase, polyphenoloxidase, proteolytic enzyme, ribonuclease, transglutaminase, xylanase, and beta-xylosidase.

### EXAMPLES

### Materials and Methods

### Strains

Aspergillus oryzae COIs1300 described in WO 2018/050666.

### Methods

General methods of PCR, cloning, cultivation etc. are well-known to a person skilled in the art and may for example be found in “Molecular cloning: A laboratory manual”, Sambrook et al. (1989), Cold Spring Harbor lab., Cold Spring Harbor, N.Y.; Ausubel, F. M. et al. (eds.); “Current protocols in Molecular Biology”, John Wiley and Sons, (1995); Harwood, C. R., and Cutting, S. M. (eds.); “DNA Cloning: A Practical Approach, Volumes I and II”, D. N. Glover ed. (1985); “Oligonucleotide Synthesis”, M. J. Gait ed. (1984); “A Practical Guide To Molecular Cloning”, B. Perbal, (1984).

### Cultivation Medium

YPM medium (2 g/I yeast extract, 2 g/I peptone, and 2% maltose).

Lipase Assay (p-Nitrophenyl Valerate)

Dilution buffer: 50 mM Tris pH 7.5, 10 mM CaCl₂), 0.1% Triton x-100; substrate stock solution: 117 μl p-Nitrophenyl valerate (sigma N4377) is diluted in 10 ml Methanol; substrate: 10 ml dilution buffer is added 100 μl substrate stock solution. 10 μl sample is added 1 ml substrate and product formation is followed by measuring the absorbance at 405 nm.

Copy Number Determination by Digital Droplet PCR (ddPCR)

Copy number variation was determined using BioRad QX200 ddPCR system per manufacturer's instructions and using the accompanying “QuantaSoft” software suite (BioRad).

Briefly, lipase gene copy number was assayed using FAM labeled Taqman probe #29 from Roche “Universal Probelibrary”. The one copy reference probe was made by Exiqon as a HEX labeled version of probe #70 from Roche “Universal Probelibrary”. The instrument allows the precise quantification a given species of DNA molecules within a given sample. Copy number is calculated by determining the ration between the one copy reference and the polynucleotide of interest.

### Example 1. Screening Intron Sequences and the Effect on Lipase Gene Expression

In order to test whether engineering of different intron sequences can be used to establish a process for the combination of different genetic elements that relies on mRNA splicing, expression of a heterologous lipase using introns was studied in A. oryzae.

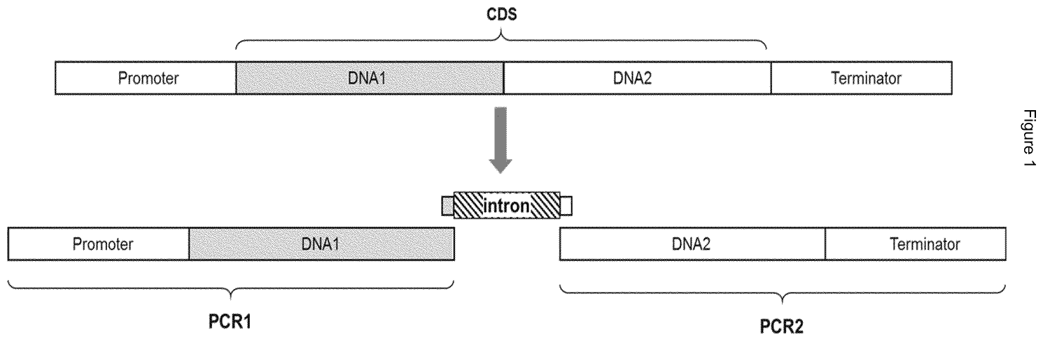

A polynucleotide sequence constituting an expression cassette, containing a promotor, a polynucleotide sequence encoding a polypeptide (lipase from Thermomyces lanuginosus, WO2016102356-A1, UniProtKB accession 059952) and a transcription terminator was used as DNA template for PCR. Two PCR products dividing the lipase coding sequence into two parts were amplified (PCR1 and PCR2, see FIG. 1). Twenty-one different introns with 5′ (gtgggcgatgtcaccggcttccttgctctc; SEQ ID NO:24) and 3′ (gacaacacgaacaaattgatcgtcctctct; SEQ ID NO:25) 30 bp flanks homologous to PCR 1-3′ and PCR2-5′, respectively, were synthesized commercially. The introns were selected among Aspergillus nidulans introns annotated in Aspergillus Genome Database, to ensure that their sequences were not homologous to A. oryzae genomic DNA (to prevent undesired homologous recombination) and that sequence length was not divisible by 3 (to ensure a frameshift if the intron did not function correctly).

Strains were constructed by mixing 500 ng of PCR1, 500 ng of PCR2 and 100 ng of each corresponding intron (specified in Table 1) using the in vivo recombination method as described in WO 2018/050666. The control strain was made by using a 60 bp fragment containing only the homologous flanks in effect inserting 0 bp.

TABLE 1
Sequences of the introns, #21 is from pyrG in A. nidulans (AN6157) and the
control #22 has no intron.

| SEQ ID | | Gene and in- |
|---|---|---|
| NO | Intron sequence | tron no. |
| 1 | GTCACCCGGGGGGTCCCGGTACGCGCGCTAAGTAG | AN2664.2, |
| | | intron 2 |
| 2 | GTATGTTCGCTTGGATTGATATTTGCGACCCCCGCTAACAG | AN3294.2, |
| | | intron 6 |
| 3 | GTATGTCCCTCTGGCACATCTG- | AN3730.2, |
| | CATCTACTTTCTAACAGAAACTAG | intron 4 |
| 4 | GTGAGTTGGTCGTCTATGAGGGTCCAACTT- | AN9390.2, |
| | GGCTCACAAGGAACAG | intron 1 |
| 5 | GTAGGTCGGATCCGCCACATATACAC- | AN4515.2, |
| | TGCGCCCGCTCATGTTGCACTAG | intron 1 |
| 6 | GTAAGATTATATCAGCCGTATAC- | AN5061.2, |
| | GAGCTGAGCGACTGACATGCATGACAG | intron 5 |
| 7 | GTGAGTACTGTCTTTTCAGCAAATGGACGACATAACTTACAA- | AN1426.2, |
| | GCTGAAAACAG | intron 3 |
| 8 | GTAAAGTCTCCCCTCTCCTCCCATCTCATGAACTCTGTAA- | AN1433.2, |
| | GCTGACCCATCCAAG | intron 2 |
| 9 | GTGAGCGCGATCTACCCGCTA- | AN5282.2, |
| | TATTGGAAGGCAATTCTGATCTCCTTGATGATAG | intron 4 |
| 10 | GTATGCTCCCCATAATCTTAGAACCTGCTGCATACTTC- | AN6324.2, |
| | TACTGACCACGATCTGTACAG | intron 1 |
| 11 | GTAAGTCTCTGCACGCGCTACGCCCAGTCAAGATTATATAAA- | AN6635.2, |
| | TACTGATATTGTATGATACATAG | intron 5 |
| 12 | GTAAGCCAGCCCGGTTGCACGGGCACCGAAATCGCCTTAC- | AN7511.2, |
| | CAGGCGCTGACACGGTCAATCGTAG | intron 1 |
| 13 | GTTTGTTAACAATCTTGATACTGCCCTCATTCTT- | AN8149.2, |
| | GCAATGTGTCACTGAATTGCTTGTGGGACAG | intron 1 |
| 14 | GTACCTTCTTTTGTATGGCTGTACGTTATTTCCT- | AN5282.2, |
| | CCCATATGGTTCCGTGTATAGGACTGAAGTCAG | intron 3 |
| 15 | GTACGTGTCTTCTTTTTTTTTGCTTGTTCTAC- | AN4006.2, |
| | CTCGCGCCTCAGTACAAGAGATACTAATTGATTTAG | intron 1 |
| 16 | GTGTGTTACCCAGGTTTCTTGCATATTCTCTCC- | AN1318.2, |
| | CGAAGTCCCTATACTCTGGCTAATCCCATATCTGCAG | intron 2 |
| 17 | GTAAGTCTTTCCACTTTCTGTCTGTGTATGTGGGG- | AN9390.2, |
| | GAAAACACATGAGTGAGCCCTTTCTGACATCTCAG | intron 2 |
| 18 | GTATGTCTTCAGGCGCTTATTGTTACCGACCTTTCCCCTT- | AN1455.2, |
| | GGAAGGAATGCTGACAGTCTTTTTCTACTCCAG | intron 1 |
| 19 | GTACGTATCATTCATGTCCTTCTACATTACGCAGACTTTGTT- | AN7135.2, |
| | GGTTGGTCGACTGACTGGTCCACTGATATAG | intron 8 |
| 20 | GTATGAAAGAGCGGCGTCCGGCCGCTGGCTGACAC- | AN6658.2, |
| | TGAATCAGACTTTGCAAGTTGCAGCAGCTAACGCCCCATAG | intron 4 |
| 21 | GTACATCCTGCACCAATGCCCCTCCAGGATAACAAA- | AN6157.2, |
| | TAGCTGATGCGTAGTGAGTACAG | intron 1 |
| 22 | No intron (Control) | |

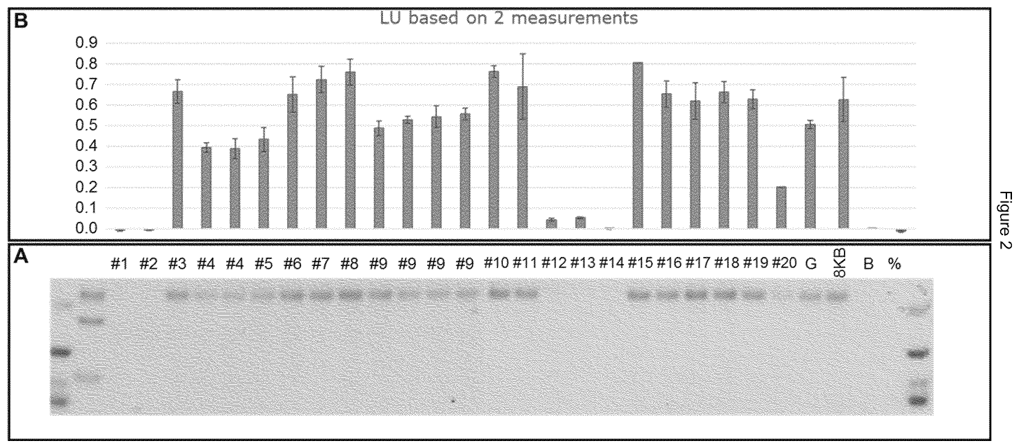

Thus, 21 different strains were constructed having a unique intron sequence placed in and thus splitting the lipase coding sequence. A control strain (#22) did not have an intron. Candidates were confirmed by sequencing to ensure that the intron sequence was placed as intended at the resident locus and ddPCR was performed to ensure that all strains contained a single copy of the lipase gene. These strains were cultivated in YPM for 5 days at 30° C. and the fermentation broth (supernatant) was filtered. The protein present in each supernatant was inspected on SDS-PAGE gels and assayed for lipase activity (FIG. 2).

As observed, most introns used were well-tolerated and had no negative effect in the overall expression level of the lipase. In fact, several of the tested introns resulted in similar or higher lipase levels compared to the control without an intron or the pyrG intron control.

This examples shows that a coding sequence of a functional polypeptide can be engineered to contain an intron and still be functional in a fungal host.

### Example 2. Using Introns to Construct Combinatorial Libraries of Genetic Elements

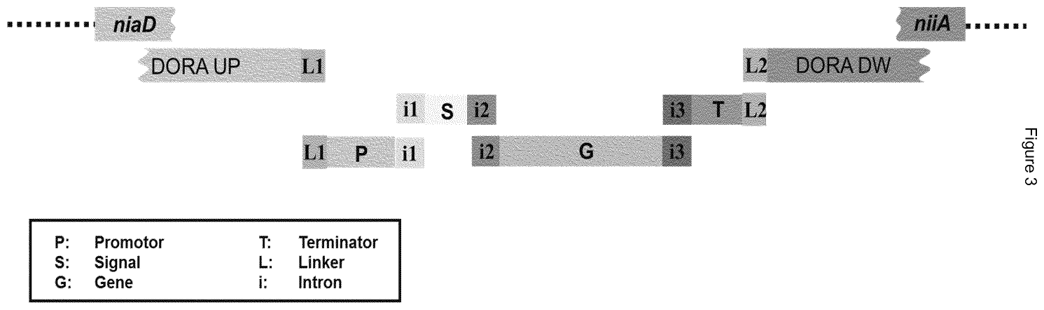

Three introns (denoted i1, i2, and i3 in the setup depicted in FIG. 3) was used to test the use of more than one intron in the construction of multiple genetic constructions and thereby the ability to assemble several DNA fragments using introns as universal linkers. As in Example 1, the functionality of the method is measured by the level of lipase protein and lipase activity in culture supernatants. Again, single copy strains were studied. In this example, three promotor sequences (P1, P2 and P3) were used as the variable cargo sequence.

The DNA fragments indicated in FIG. 3 (DORA UP, P, S, G, T, and DORA DW) were generated by PCR. The oligonucleotides used contained tails having the 30 bp linking overlaps specified in Table 2. DORA UP and DORA DW are standard fragments used for in vivo recombination method into strain COIs1300 as described in WO 2018/050666. Three different promotors, P1-P3, were used in three experiments to make 3 different types of strains, differing each only in the chosen promotor. The promoters were selected from standard fungal promoters. The fragments were mixed using 500 ng of DORA UP and DW fragments (Fragments 1 and 6, Table 3) and the specified amounts for Fragments 2-5 (Table 3).

TABLE 2
The nucleotide sequences of the linking sequences. The sequences i1-i3
are introns that were found to result in production of functional lipase
in Example 1.

| Code | Sequence | Description |
|---|---|---|
| L1 | cctaactgcgctgagggtttacgcgcctga | Linker 1 |
| | | (SEQ ID NO: 22) |
| i1 | GTAAAGTCTCCCCTCTCCTCCCATCTCATGAACTCTGTAA- | SEQ ID NO: 8 |
| | GCTGACCCATCCAAG | |
| 12 | GTGAGTACTGTCTTTTCAGCAAATGGACGACATAACTTACAA- | SEQ ID NO. 7 |
| | GCTGAAAACAG | |
| i3 | GTGTGTTACCCAGGTTTCTTGCATATTCTCTCCCGAAGTCCC- | SEQ ID NO: 16 |
| | TATACTCTGGCTAATCCCATATCTGCAG | |
| L2 | gaaacctgaggcaacaagggggcgcgatttacc | Linker 2 |
| | | (SEQ ID NO: 23) |

TABLE 3
For each of the three experiments the six DNA fragments
(F) were mixed using the amounts specified.

| Exp. | Fl | ng | F2 | ng | F3 | ng | F4 | ng | F5 | ng | F6 | ng |
|---|---|---|---|---|---|---|---|---|---|---|---|---|
| 1 | DORA | 500 | P1 | 150 | Signal | 150 | Lipase | 700 | Terminator | 140 | DORA | 500 |
| | UP | | | | | | | | | | DW | |
| 2 | DORA | 500 | P2 | 150 | Signal | 150 | Lipase | 700 | Terminator | 140 | DORA | 500 |
| | UP | | | | | | | | | | DW | |
| 3 | DORA | 500 | P3 | 250 | Signal | 150 | Lipase | 700 | Terminator | 140 | DORA | 500 |
| | UP | | | | | | | | | | DW | |

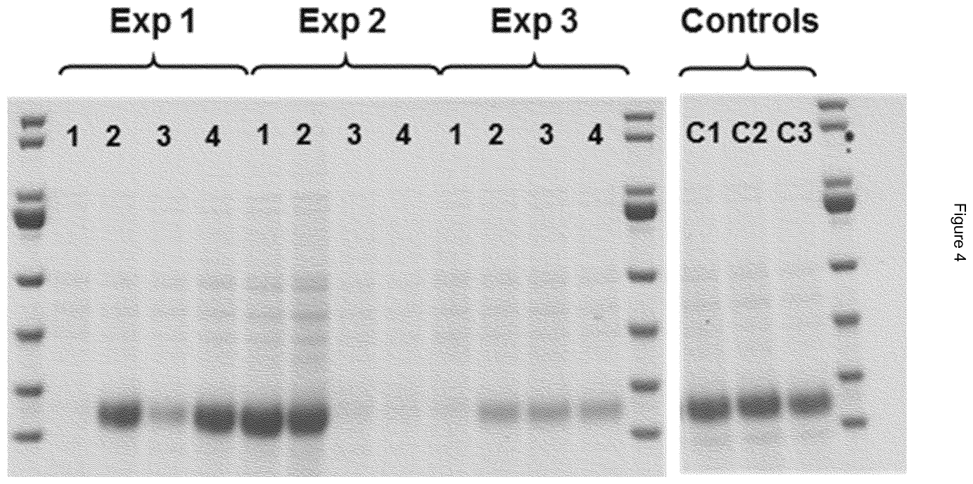

From each experiment, four transformants were selected and cultivated in YPM medium for five days at 30° C. and the culture supernatant was filtered. The samples were run on SDS-PAGE gels as seen in FIG. 4.

As shown, assembly of six fragments using introns was successful with three different promotor fragments. In all cases, the presence of lipase was observed in the supernatants. Thus, several DNA fragments can be effectively assembled using introns as universal linkers within the coding region of the gene of interest.

### Example 3. Introns are Functional for Protein Expression in Multicopy Strains

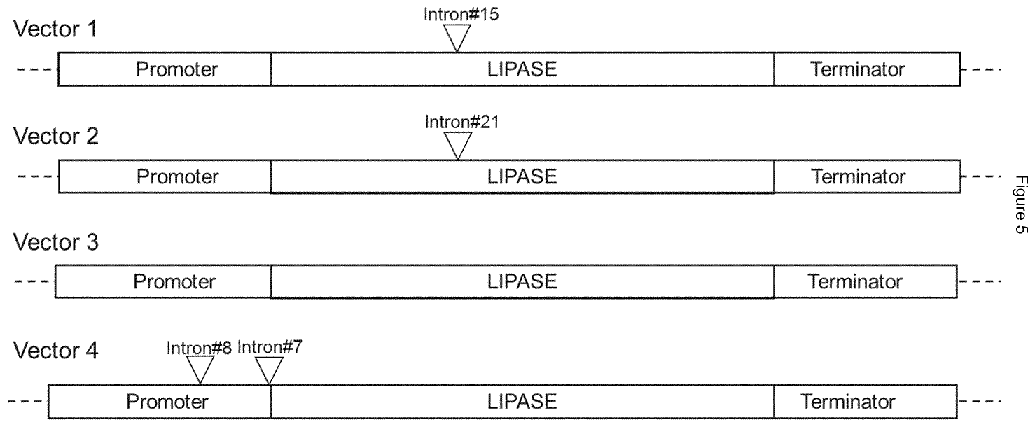

Expression vectors were constructed as disclosed in WO 2013/178674. Briefly, vectors are constructed that integrate in a multicopy fashion into a single specific locus in the A. oryzae genome, restoring a selective gene. The copy number of the lipase gene in the selected strains can be determined by ddPCR. Four vectors were constructed by standard cloning methods in E. coli (FIG. 5).

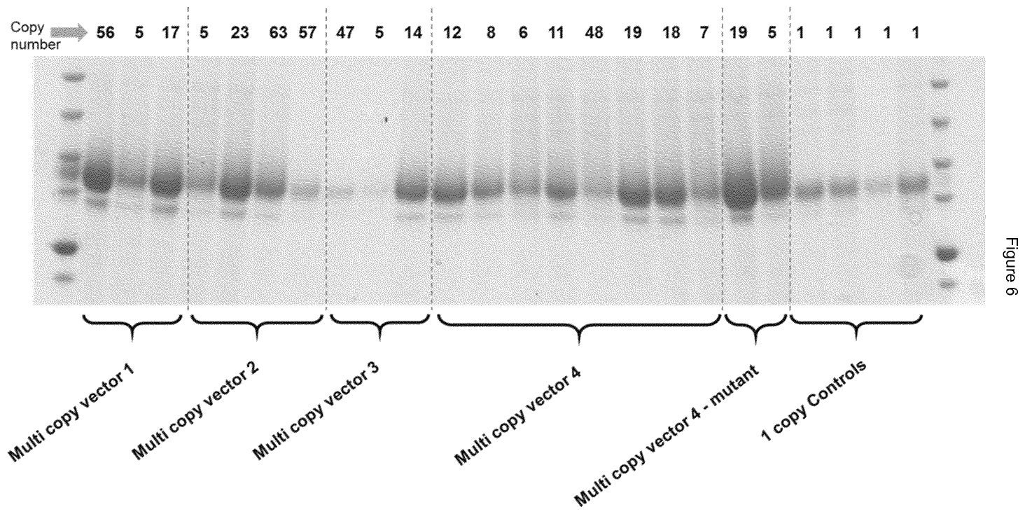

Transformants from each vector were selected and cultivated in YPM for five days at 30° C. and supernatants were run on SDS-PAGE gels (FIG. 6). The copy number of the lipase gene in each of the selected transformants was determined by ddPCR. The results showed that the functionality of multiple introns is maintained even at high copy numbers. Thus, the use of multiple introns enables production of functional lipase thereby enabling the construction of multiple strains with combinations of genetic elements.

Additionally, sequence verification revealed that a version of vector 4 (FIG. 5) which had a single base deletion in the intron sequence from 5′-GTAAAGTCTCCCCTCTCCTCCCATCTCATGAACTCTGTAAGCTGACCCATCCAAG-3′ to 5′-GAAAGTCTCCCCTCTCCTCCCATCTCATGAACTCTGTAAGCTGACCCATCCAAG-3′, called Vector 4—mutant, had been obtained. Comparison between the wt and the mutant intron sequence indicates better expression of lipase in the latter as judged by the 19-copy strain derived by using the two vectors (FIG. 6). Therefore, sequence modification of the intervening introns leads to increased lipase yields.

Moreover, this Example demonstrates that the functionality of introns is not limited by a low copy number, and that using introns as linkers also works for expression in production-relevant strains.

### Example 4. Multiple Introns and Promotor Library

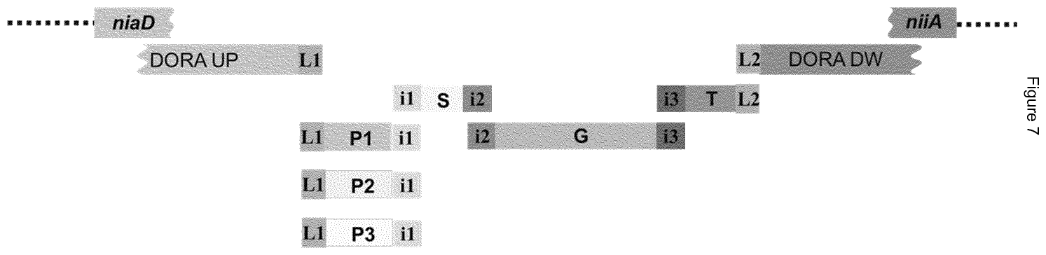

This example is similar to Example 2 described above except that three promotor fragments are added to the mix simultaneously (FIG. 7).

TABLE 4
DNA fragments (F) 1, 3, 4, 5, and 6 were mixed with all three types of F2 allowing
any one of the three promotors to be integrated during the transformation.
The fragments were mixed using the amounts specified.

| Exp. | F1 | ng | F2 | ng | F3 | ng | F4 | ng | F5 | ng | F6 | ng |
|---|---|---|---|---|---|---|---|---|---|---|---|---|
| 1 | DORA | 500 | P1 | 50 | Signal | 150 | Lipase | 700 | Terminator | 140 | DORA | 500 |
| | UP | | | | | | | | | | DW | |
| | | | P2 | 50 | | | | | | | | |
| | | | P3 | 80 | | | | | | | | |

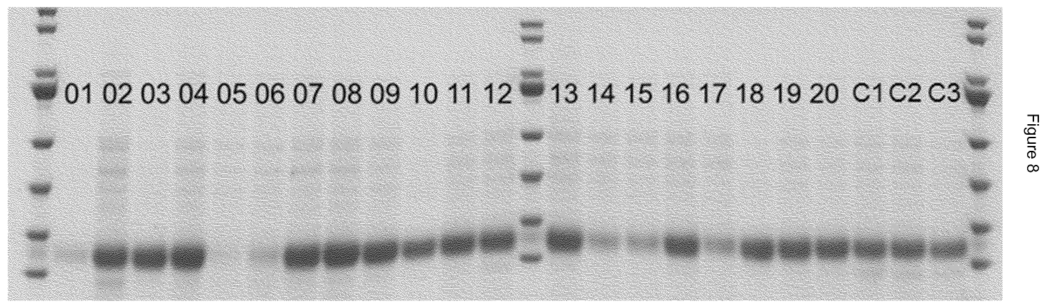

32 transformants from the simultaneous experiment were selected and cultivated in YPM medium for five days at 30° C. and the supernatants were run on SDS-PAGE gels (FIG. 8). Subsequent sequencing of the strains revealed that all 3 three types of promotor were represented in this limited sample size. Thus, the example demonstrates that the intron linkers can be used to generate combinatorial libraries.

### Example 5. The Polynucleotide Sequence within Introns is Flexible

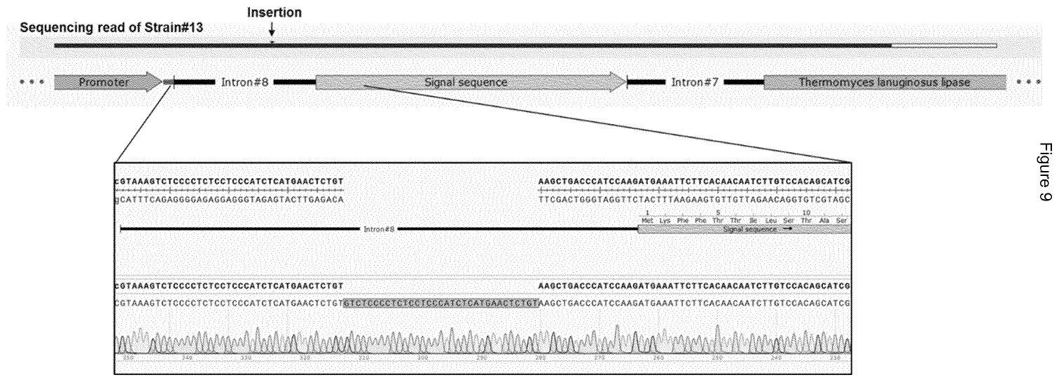

DNA sequencing of strain number thirteen from Example 4 revealed that the sequence of intron #8 (SEQ ID NO: 8) had been mutated during the assembly of the fragments resulting in a novel functional intron. The novel intron (FIG. 9) is 33 nucleotides larger than intron #8 (SEQ ID NO: 8), but as seen in FIG. 8, strain #13 is proficiently expressing protein indicating that the novel intron is fully functional.

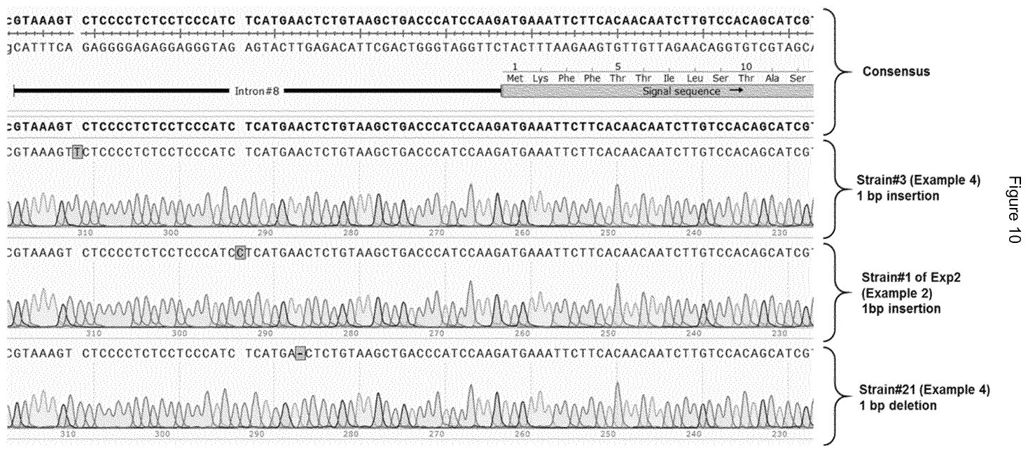

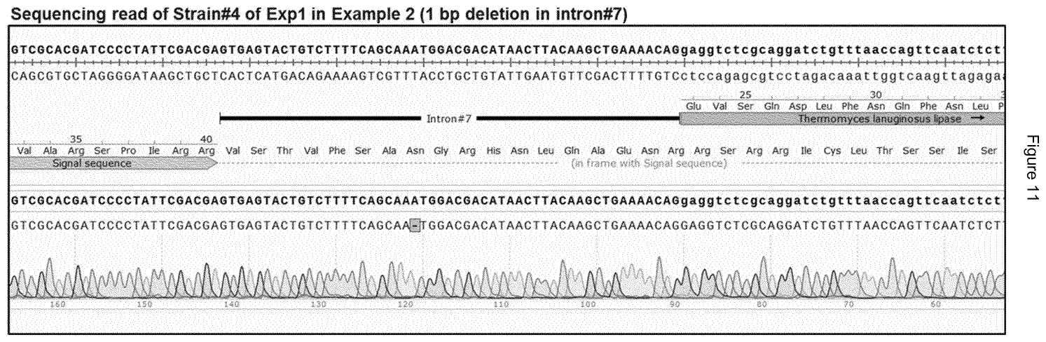

More examples of sequence smaller variations within introns have been observed for intron #8 (SEQ ID NO: 8, FIG. 10) and intron #7 (SEQ ID NO: 7, FIG. 11), indicating that introns are tolerant of modification within the sequence.

### Example 6. Matrix Cloning of Codon Variants Using Introns as Linkers

Using the same methods applied in Examples 2 and 4, a matrix was set up to clone six different codon variants of Thermomyces lanuginosus lipase (UniProtKB accession 059952). Briefly, fragments (Table 5) were amplified by PCR and gel purified.

TABLE 5
The table shows the fragments (F) needed for the
matrix experiment. Oligo #1 and oligo #2
were the PCR primers used and
the size of the resulting fragments
are shown in base pairs (bp). The amounts
of DNA needed of each fragment
for one transformation is shown in ng.

| | | | | | Amounts | Reactions | Total |
|---|---|---|---|---|---|---|---|
| F | Name | oligo #1 | oligo #2 | Size (bp) | (ng) | needed | DNA (ng) |
| 1 | DORA UP | 2427 | 2564 | 2760 | 200 | 13 | 2860 |
| 2 | DORA DW | 2530 | 2430 | 2690 | 200 | 13 | 2860 |
| 4 | Promotor | 2533 | 2534 | 820 | 150 | 13 | 2145 |
| 15 | Lipase variant #15 | 2545 | 2546 | 965 | 200 | 2 | 440 |
| 19 | Terminator | 2551 | 2552 | 772 | 150 | 13 | 2145 |
| 23 | Lipase variant #23 | 2545 | 2546 | 906 | 200 | 2 | 440 |
| 24 | Lipase variant #24 | 2545 | 2546 | 906 | 200 | 2 | 440 |
| 25 | Lipase variant #25 | 2545 | 2546 | 906 | 200 | 2 | 440 |
| 26 | Lipase variant #26 | 2559 | 2560 | 906 | 200 | 2 | 440 |
| 27 | Lipase signal | 2588 | 2589 | 227 | 40 | 13 | 573 |
| 38 | Lipase variant #38 | 2545 | 2597 | 520 | 100 | 2 | 220 |
| | 1st half | | | | | | |
| 39 | Lipase variant #38 | 2598 | 2546 | 427 | 100 | 2 | 220 |
| | 2nd half | | | | | | |

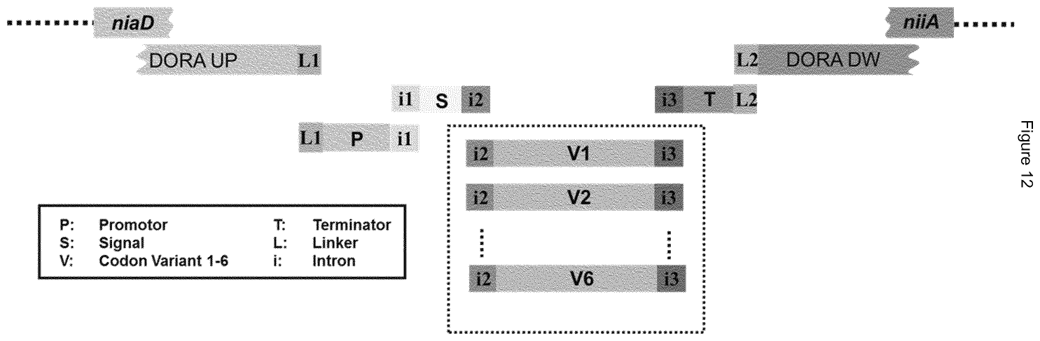

Transformations were performed as described in WO 2018/050666 using the fragments specified in Table 6 in the amounts shown in Table 5. Thus, each transformation mix consisted of the 6 fragments specified in Table 6, except transformation Lip #7 (the negative control) where no DNA was used as the gene variant. A graphical illustration of the matrix is shown in FIG. 12.

TABLE 6
The table shows the matrix used for the 7 transformations. The numbers in the categories
“UP”, “Promotor”, “Signal”, “Variant”, “Terminator”, and “Down” represent the fragment
number from Table 5. Fragment 38 and 39 were partial overlapping fragments of
lipase that could combine to form a complete gene, so in the case of Lip #6, seven
fragments were added to the transformation.

| Transformation | UP # | Promotor # | Signal # | Variant # | terminator # | Down # |
|---|---|---|---|---|---|---|
| Lip #1 | 1 | 4 | 27 | 15 | 19 | 2 |
| Lip #2 | 1 | 4 | 27 | 23 | 19 | 2 |
| Lip #3 | 1 | 4 | 27 | 24 | 19 | 2 |
| Lip #4 | 1 | 4 | 27 | 25 | 19 | 2 |
| Lip #5 | 1 | 4 | 27 | 26 | 19 | 2 |
| Lip #6 | 1 | 4 | 27 | 38 & 39 | 19 | 2 |
| Lip #7 | 1 | 4 | 27 | No DNA | 19 | 2 |

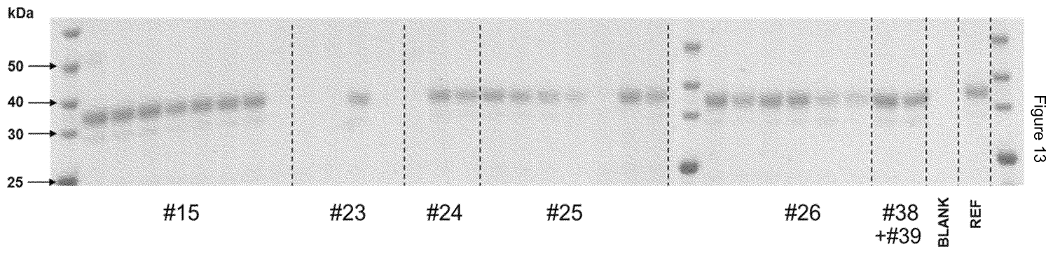

Transformants were obtained for each gene variant. These were selected and cultivated in YPM medium for five days at 30° C. and the supernatants were run on SDS-PAGE gels (FIG. 13). Rewardingly, each lipase variant was represented in the transformants.

### REFERENCES

Cerqueira G C, Arnaud M B, Inglis D O, Skrzypek M S, Binkley G, Simison M, Miyasato S R, Binkley J, Orvis J, Shah P, Wymore F, Sherlock G, Wortman J R (2014). The Aspergillus Genome Database: multispecies curation and incorporation of RNA-Seq data to improve structural gene annotations. Nucleic Acids Res 42 (1); D705-10.
Arnaud M B, Cerquiera G C, Inglis D O, Skrzypek M S, Binkley J, Shah P, Wymore F, Binkley G, Miyasato S R, Simison M, Wortman J R, Sherlock G. “Aspergillus Genome Database” http://www.aspergillusgenome.org/ (20160416).

1. A nucleic acid construct comprising in a 5′ to 3′ direction a promoter, a first intron, a polynucleotide encoding a polypeptide of interest, a second intron, and a transcription terminator, wherein the promoter, the first intron, the polynucleotide encoding a polynucleotide of interest, the second intron, and the transcription terminator are operably linked.

2. The nucleic acid construct according to claim 1, which further comprises a polynucleotide encoding a signal peptide and a third intron, wherein the polynucleotide encoding a signal peptide and the third intron are operably linked to and located between the first intron and the polynucleotide encoding a polypeptide of interest.

3. The nucleic acid construct according to claim 1, wherein the promoter is a heterologous promoter; preferably the promoter is a fungal promoter.

4. The nucleic acid construct according to claim 1, wherein the polypeptide of interest comprises or consists of an enzyme; preferably the enzyme is selected from the group consisting of hydrolase, isomerase, ligase, lyase, oxidoreductase, or transferase; more preferably the enzyme is selected from the group consisting of aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, nuclease, oxidase, pectinolytic enzyme, peroxidase, phosphodiesterase, phytase, polyphenoloxidase, proteolytic enzyme, ribonuclease, transglutaminase, xylanase, and beta-xylosidase.

5. The nucleic acid construct according to claim 1, wherein the introns are different and individually comprise no more than 200 nucleotides, i.e., no more than 175, 150, 125, 100, 90, 80, 70, 60, or 50 nucleotides.

6. The nucleic acid construct according to claim 1, wherein the introns are individually selected from the group consisting of SEQ ID NO: 1-21.

7. The nucleic acid construct according to claim 1, wherein the first intron is located between the promoter and the start codon of the polynucleotide encoding a polypeptide of interest, and wherein the second intron is located between the stop codon of the polynucleotide encoding a polypeptide of interest and the transcription terminator.

8. An expression vector comprising a nucleic acid construct according to claim 1.

9. A eukaryotic host cell comprising in its genome a nucleic acid construct according to claim 1.

10. The eukaryotic host cell according to claim 9, said host cell being a mammalian, plant, or fungal host cell.

11. The eukaryotic host cell of claim 10, wherein the fungal host cell is a yeast host cell.

12. The eukaryotic host cell of claim 10, wherein the fungal host cell is a filamentous fungal host cell.

13. A method for constructing a eukaryotic host cell, the method comprising transforming a eukaryotic cell with:
a) a first polynucleotide comprising in a ′5 to 3 direction a promoter and a first DNA sequence;
b) a second polynucleotide comprising in a ′5 to ′3 direction a second DNA sequence, a coding sequence of a polypeptide of interest, and a third DNA sequence; and
c) a third polynucleotide comprising in a ′5 to 3′ direction a fourth DNA sequence and a transcription terminator;
wherein the first, second, and third polynucleotides are operably linked, wherein the first and second DNA sequences and the third and fourth DNA sequences are pairwise capable of homologous recombination and subsequent formation of introns, and wherein the resulting introns are capable of RNA splicing upon transcription.

14. A method according to claim 13, wherein the host cell is further transformed with a fourth polynucleotide comprising in a 5′ to 3′ direction a fifth DNA element, a polynucleotide encoding a signal peptide, and a sixth DNA element;
wherein the first, second, third, and fourth polynucleotides are operably linked, wherein the first and fifth DNA sequences, the sixth and second DNA sequences, and the third and fourth DNA sequences are pairwise capable of homologous recombination and subsequent formation of introns, and wherein the resulting introns are capable of RNA splicing upon transcription.

15. A method for producing a polypeptide of interest, the method comprising the steps of:
a) providing a eukaryotic host cell according to claim 9;
b) cultivating said host cell under conditions conducive for expression of the polypeptide of interests; and, optionally
c) recovering the polypeptide of interest.

16. The eukaryotic host cell according to claim 9, said host cell being a fungal host cell.

17. The eukaryotic host cell of claim 10, wherein the fungal host cell is a yeast host selected from Candida, Hansenula, Kluyveromyces, Pichia, Saccharomyces, Schizosaccharomyces, and Yarrowia.

18. The eukaryotic host cell of claim 10, wherein the fungal host cell is a yeast host selected from Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, and Yarrowia lipolytica.

19. The eukaryotic host cell of claim 10, wherein the fungal host cell is a filamentous fungal host cell selected from Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, and Trichoderma.

20. The eukaryotic host cell of claim 10, wherein the fungal host cell is a filamentous fungal host cell selected from Aspergillus niger, Aspergillus oryzae, Fusarium venenatum, and Trichoderma reesei.


SEQUENCE LISTING (33 sequences)

SEQ ID NO: 1 - DNA, 35 bp, Aspergillus nidulans
gtcacccggggggtcccggtacgcgcgctaagtag

SEQ ID NO: 2 - DNA, 41 bp, Aspergillus nidulans
gtatgttcgcttggattgatatttgcgacccccgctaacag

SEQ ID NO: 3 - DNA, 46 bp, Aspergillus nidulans
gtatgtccctctggcacatctgcatctactttctaacagaaactag

SEQ ID NO: 4 - DNA, 46 bp, Aspergillus nidulans
gtgagttggtcgtctatgagggtccaacttggctcacaaggaacag

SEQ ID NO: 5 - DNA, 49 bp, Aspergillus nidulans
gtaggtcggatccgccacatatacactgcgcccgctcatgttgcactag

SEQ ID NO: 6 - DNA, 50 bp, Aspergillus nidulans
gtaagattatatcagccgtatacgagctgagcgactgacatgcatgacag

SEQ ID NO: 7 - DNA, 53 bp, Aspergillus nidulans
gtgagtactgtcttttcagcaaatggacgacataacttacaagctgaaaacag

SEQ ID NO: 8 - DNA, 55 bp, Aspergillus nidulans
gtaaagtctcccctctcctcccatctcatgaactctgtaagctgacccatccaag

SEQ ID NO: 9 - DNA, 55 bp, Aspergillus nidulans
gtgagcgcgatctacccgctatattggaaggcaattctgatctccttgatgatag

SEQ ID NO: 10 - DNA, 59 bp, Aspergillus nidulans
gtatgctccccataatcttagaacctgctgcatacttctactgaccacgatctgtacag

SEQ ID NO: 11 - DNA, 65 bp, Aspergillus nidulans
gtaagtctctgcacgcgctacgcccagtcaagattatataaatactgatattgtatgatacatag

SEQ ID NO: 12 - DNA, 65 bp, Aspergillus nidulans
gtaagccagcccggttgcacgggcaccgaaatcgccttaccaggcgctgacacggtcaatcgtag

SEQ ID NO: 13 - DNA, 65 bp, Aspergillus nidulans
gtttgttaacaatcttgatactgccctcattcttgcaatgtgtcactgaattgcttgtgggacag

SEQ ID NO: 14 - DNA, 67 bp, Aspergillus nidulans
gtaccttcttttgtatggctgtacgttatttcctcccatatggttccgtgtataggactgaagtcag

SEQ ID NO: 15 - DNA, 68 bp, Aspergillus nidulans
gtacgtgtcttctttttttttgcttgttctacctcgcgcctcagtacaagagatactaattgatttag

SEQ ID NO: 16 - DNA, 70 bp, Aspergillus nidulans
gtgtgttacccaggtttcttgcatattctctcccgaagtccctatactctggctaatcccatatctgcag

SEQ ID NO: 17 - DNA, 70 bp, Aspergillus nidulans
gtaagtctttccactttctgtctgtgtatgtgggggaaaacacatgagtgagccctttctgacatctcag

SEQ ID NO: 18 - DNA, 73 bp, Aspergillus nidulans
gtatgtcttcaggcgcttattgttaccgacctttccccttggaaggaatgctgacagtctttttctactccag

SEQ ID NO: 19 - DNA, 73 bp, Aspergillus nidulans
gtacgtatcattcatgtccttctacattacgcagactttgttggttggtcgactgactggtccactgatatag

SEQ ID NO: 20 - DNA, 76 bp, Aspergillus nidulans
gtatgaaagagcggcgtccggccgctggctgacactgaatcagactttgcaagttgcagcagctaacgccccatag

SEQ ID NO: 21 - DNA, 59 bp, Aspergillus nidulans
gtacatcctgcaccaatgcccctccaggataacaaatagctgatgcgtagtgagtacag

SEQ ID NO: 22 - DNA, 30 bp, Artificial Sequence
  Linker 1
cctaactgcgctgagggtttacgcgcctga

SEQ ID NO: 23 - DNA, 33 bp, Artificial Sequence
  Linker 2
gaaacctgaggcaacaagggggcgcgatttacc

SEQ ID NO: 24 - DNA, 30 bp, Artificial sequence
  30 bp flanks
gtgggcgatgtcaccggcttccttgctctc

SEQ ID NO: 25 - DNA, 30 bp, Artificial sequence
  30 bp flanks
gacaacacgaacaaattgatcgtcctctct

SEQ ID NO: 26 - DNA, 91 bp, Artificial sequence
  Intron #8
gtaaagtctcccctctcctcccatctcatgaactctgtaagctgacccatccaatatgaaattcttcacaacaatcttgtccacagcatcg

SEQ ID NO: 27 - DNA, 124 bp, Artificial sequence
  Intron #8 with insert
gtaaagtctcccctctcctcccatctcatgaactctgtgtctcccctctcctcccatctcatgaactctgtaagctgacccatccaatatgaaattcttcacaacaatcttgtccacagcatcg

SEQ ID NO: 28 - DNA, 91 bp, Artificial sequence
  Consensus
gtaaagtctcccctctcctcccatctcatgaactctgtaagctgacccatccaagatgaaattcttcacaacaatcttgtccacagcatcg

SEQ ID NO: 29 - DNA, 92 bp, Artificial sequence
  Strain#3 (Example 4) 1 bp insertion
gtaaagttctcccctctcctcccatctcatgaactctgtaagctgacccatccaagatgaaattcttcacaacaatcttgtccacagcatcg

SEQ ID NO: 30 - DNA, 92 bp, Artificial sequence
  Strain#1 of Exp2 (Example 2) 1 bp insertion
gtaaagtctcccctctcctcccatcctcatgaactctgtaagctgacccatccaagatgaaattcttcacaacaatcttgtccacagcatcg

SEQ ID NO: 31 - DNA, 90 bp, Artificial sequence
  Strain#21 (Example 4) 1 bp deletion
gtaaagtctcccctctcctcccatctcatgactctgtaagctgacccatccaagatgaaattcttcacaacaatcttgtccacagcatcg

SEQ ID NO: 32 - DNA, 114 bp, Artificial sequence
  Strai#4 of Exp1 in Example 2 intron#7
gtcgcacgatcccctattcgacgagtgagtactgtcttttcagcaaatggacgacataacttacaagctgaaaacaggaggtctcgcaggatctgtttaaccagttcaatctct

SEQ ID NO: 33 - DNA, 113 bp, Artificial sequence
  Strai#4 of Exp1 in Example 2 intron#7 with 1 bp deletion
gtcgcacgatcccctattcgacgagtgagtactgtcttttcagcaatggacgacataacttacaagctgaaaacaggaggtctcgcaggatctgtttaaccagttcaatctct